(19) United States
(12) Patent Application Publication
(10) Pub. No.: US 2018/0199810 A1
(43) Pub. Date: Jul. 19, 2018

(54) SYSTEMS AND METHODS FOR PUPILLARY DISTANCE ESTIMATION FROM DIGITAL FACIAL IMAGES

(71) Applicant: Smart Vision Labs, Inc., New York, NY (US)

(72) Inventor: Kaccie Y. Li, New York, NY (US)

(21) Appl. No.: 15/871,874

(22) Filed: Jan. 15, 2018

Related U.S. Application Data
(60) Provisional application No. 62/446,403, filed on Jan. 14, 2017.

(51) Int. Cl. A61B 3/11; G06T 7/60; G06T 7/593; G06T 7/11; G06T 7/80; A61B 3/14

(52) U.S. Cl. CPC A61B 3/111; G06T 7/60; G06T 7/593; G06T 7/11; G06T 2207/30204; A61B 3/14; G06T 2207/30041; G06T 2207/10012; G06T 2207/10028; G06T 7/85

(57) ABSTRACT

The present disclosure relates to systems and methods for measuring a pupillary distance of a patient. In one embodiment, a method comprises identifying a first pupil and a second pupil within an image of a face of the patient; computing an eyes-to-camera distance; computing a rotational angle of the first pupil or the second pupil; and computing the pupillary distance based on the eyes-to-camera distance and the rotational angle.

### TECHNICAL FIELD

Embodiments of the present disclosure relate to patient eye examinations, and, specifically, to systems and methods for performing pupillary distance measurements.

### BACKGROUND

Pupillary distance is the distance between the centers of the pupils of each eye, and is used in preparing prescription eyeglasses. Current methods of pupillary distance measurement often require intervention of trained personnel. Moreover, although efforts have been made to automate the process, such methods are not fully automated as they still require the distance between the patient and camera to be fixed and known at the time of image capture.

### BRIEF DESCRIPTION OF THE DRAWINGS

The embodiments of the present disclosure are illustrated by way of example and not by way of limitation, and will become apparent upon consideration of the following description, taken in conjunction with the accompanying drawings, in which:

FIG. 1 illustrates an exemplary pupillary measurement performed by a patient in accordance with an embodiment of the present disclosure;

FIG. 2 illustrates exemplary patterns for use in data calibration in accordance with an embodiment of the present disclosure;

FIG. 3A illustrates an exemplary pupillary measurement performed by an examiner/technician in accordance with an embodiment of the present disclosure;

FIG. 3B illustrates an exemplary pupillary measurement performed by a patient in accordance with another embodiment of the present disclosure;

FIG. 4 is a flow diagram illustrating a method for performing a pupillary distance measurement in accordance with various embodiments of the present disclosure;

FIG. 5A is a flow diagram illustrating a method for computing a pupillary distance in accordance with various embodiments of the present disclosure;

FIG. 5B is a schematic illustrating a similar triangle relationship used for determining eye rotational angle in accordance with an embodiment of the present disclosure;

FIG. 6 illustrates an inner iris boundary, an outer iris boundary, and a corneal diameter;

FIG. 7 illustrates image capture based on stereo vision computations in accordance with an embodiment of the present disclosure;

FIG. 8 illustrates the use of structured light imaging in accordance with an embodiment of the present disclosure; and

FIG. 9 is an illustrative computer system that certain embodiments of the disclosure may utilize.

### DETAILED DESCRIPTION

The following description and drawings referenced herein illustrate embodiments of this application's subject matter, and are not intended to limit the scope. Those of ordinary skill in the art will recognize that other embodiments of the disclosed systems, devices, and methods are possible. All such embodiments should be considered within the scope of the claims. Reference numbers are not necessarily discussed in the order of their appearances in the drawings. Depictions of various components within the drawings, such as optical components, are illustrative and not necessarily drawn to scale.

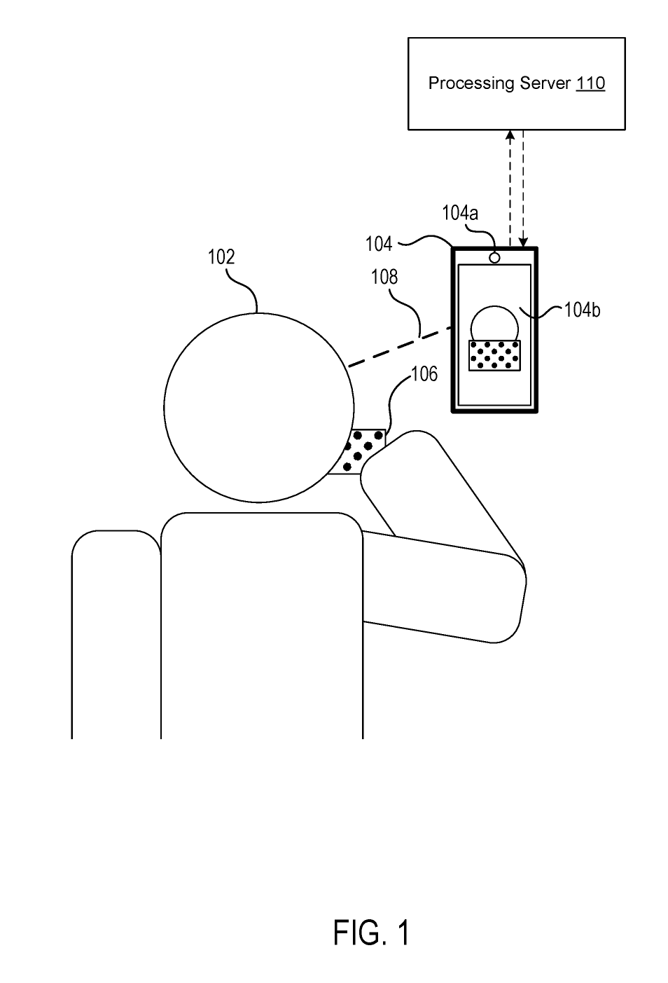

The embodiments described herein relate to systems and methods for performing automated pupillary distance (PD) measurements. FIG. 1 illustrates an exemplary pupillary measurement performed by a patient 102 in accordance with embodiments of the present disclosure. A front-facing camera 104a of a user device 104 may be used for facial image capture, with a live video feed and/or captured image displayed by an integrated display screen 104b. In some embodiments, the user device 104 may be mounted with a front-facing camera of the user device 104 facing the patient 102 as illustrated.

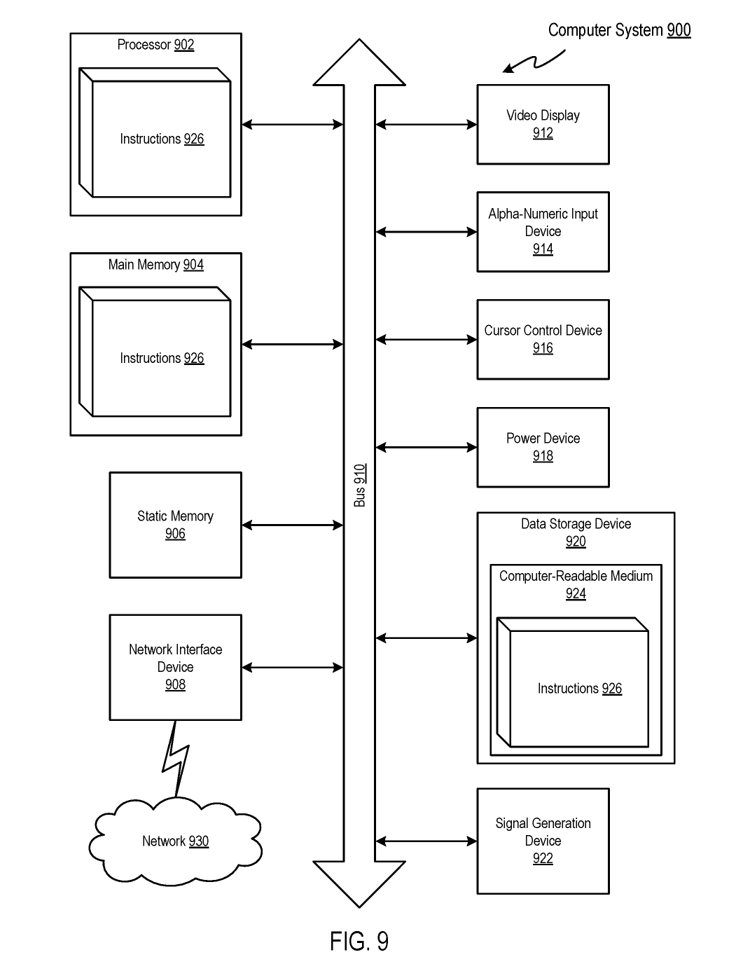

The user device 104 may be, for example, a handheld device (such as a smart phone), a laptop computer having an integrated or separate webcam, or any other device suitable for capturing visual images. As used herein, “user device” may refer to a smartphone, a mobile phone, a personal digital assistant, a personal computer, a laptop, a netbook, a tablet computer, a palmtop computer, a television (e.g., a “smart TV”), or any device having a built-in camera. A user device may also refer to a portable camera or an optical imaging device operatively coupled to a computing device (e.g., a webcam, Amazon's DeepLens, etc.). Smartphones are mobile phones having a computer, an illuminated screen, and a camera, among other features. Other user devices having a camera may be used in accordance with the subject matter of this application. For example, a user device that may be used in accordance with the disclosed embodiments could be a phone (or smartphone) equipped with a camera, although other devices such as tablet computers, laptop computers, certain audio or video players, and ebook readers may also be used, any of which may include a light detector (e.g., a camera) and either a processing device or a transceiver for communicating the information captured by the camera to another device with a processing device. General computer devices that may serve as user devices are described in more detail with respect to FIG. 9.

Referring once again to FIG. 1, the dotted line 108 conveys that the user device 104 is mounted at roughly the same level as the patient's eyes. In certain embodiments, the camera of the user device 104 is at eye-level with the patient's eyes, and an indication may be provided by the user device 104 to the patient to look directly at the camera while the facial image is captured. In certain embodiments, the camera's field-of-view (FOV) may be either static or frozen, which is common for the simple front-facing cameras in most user devices.

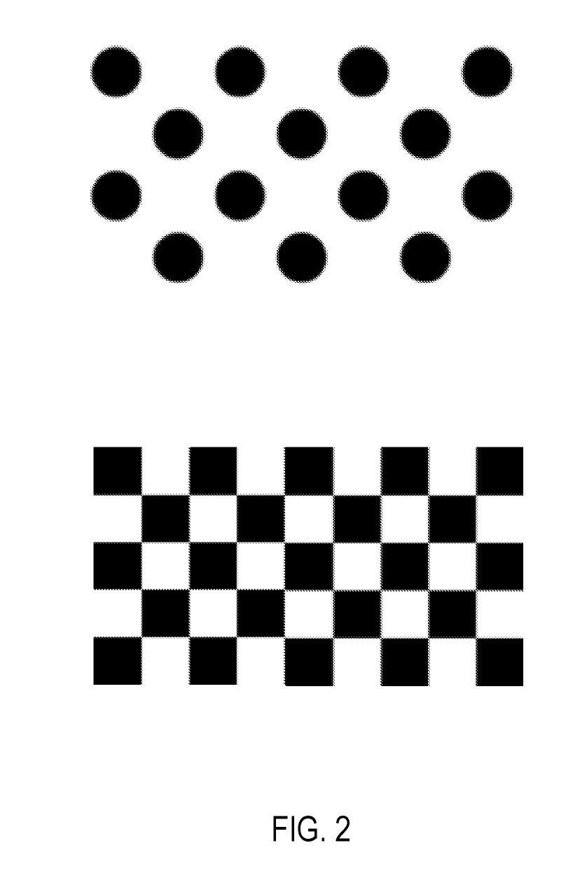

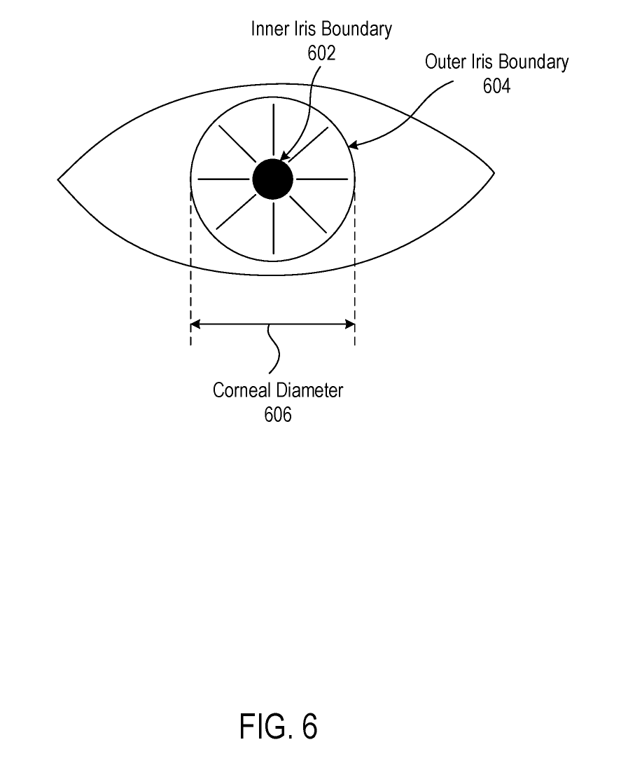

In certain embodiments, a patterned object 106 may be held by the patient 102 adjacent to his/her face during image capture. For example, the patterned object may be held by the patient 106 over his/her mouth in contact with the upper lip and roughly square with the camera plane. In other embodiments, the patterned object 106 may be placed at another location of the patient's face. In certain embodiments, the patterned object is any object having a pattern that may be recognizable by an algorithm in which from which physical scale data can be extracted (e.g., distance calibration can be performed by identifying features within the pattern that have known dimensions). In certain embodiments, the patterned object may be a card, such as a standard ID-1 format card. The card may have any suitable pattern thereon, such as a standard camera calibration pattern. Examples of standard camera calibration patterns are shown in FIG. 2, which include a hexagonal grid of black circles and a checkerboard pattern. The circle pattern in FIG. 2, for example, has 2 rows and 7 columns of dots, which may be known in advance prior to any image processing so that a pattern-finding algorithm is able to identify it within a captured image. An accurately isolated pattern may serve as the scale reference to determine the physical size represented by each pixel in the image. In certain embodiments, other patterns may be used, including text with known font dimensions. In certain embodiments, a scale object may be used in lieu of the patterned object, which may have a particular shape and size that an image processing algorithm may recognize for calibration purposes. In certain embodiments, the patterned object may be a scale object (e.g., a scale object having a pattern formed thereon). In certain embodiments, as will be discussed with respect to FIGS. 6-8, the patterned object 106 may be omitted.

Using a custom-patterned ID-1 format card has advantages over other types of patterned objects, such as credit cards or driver's licenses (which are compatible with the embodiments described herein), in that the object-recognition algorithm problem is greatly simplified, and such cards avoid potential issues with regard to the disclosure of sensitive information. An asymmetric or hexagonal dot pattern, in particular, and its corresponding detection algorithm is one of many solutions to this pattern-algorithm pair approach of providing a scale for estimating physical sizes of objects in the image. In other embodiments, wire grids (e.g. ronchi ruling), checker-boards, stripes, a United States Air Force (USAF) target, or any other pattern/method used for camera calibration may be used.

In certain embodiments, the user device 104 may process the captured facial image data via a standalone application implemented by the user device 104. In other embodiments, the user device may capture the facial image data (e.g., using a web browser interface) and then transmit the facial image data, via a communications network, to a processing server 110 for processing the facial image data. The processing server may be a local server or a remote server that may subsequently transmit the results of the processing back to the user device 104 or to another device (e.g., a clinician's device). In certain embodiments, the processing server 110 may be omitted. In such embodiments, image processing, as discussed herein, may be performed by the user device 104 or another device.

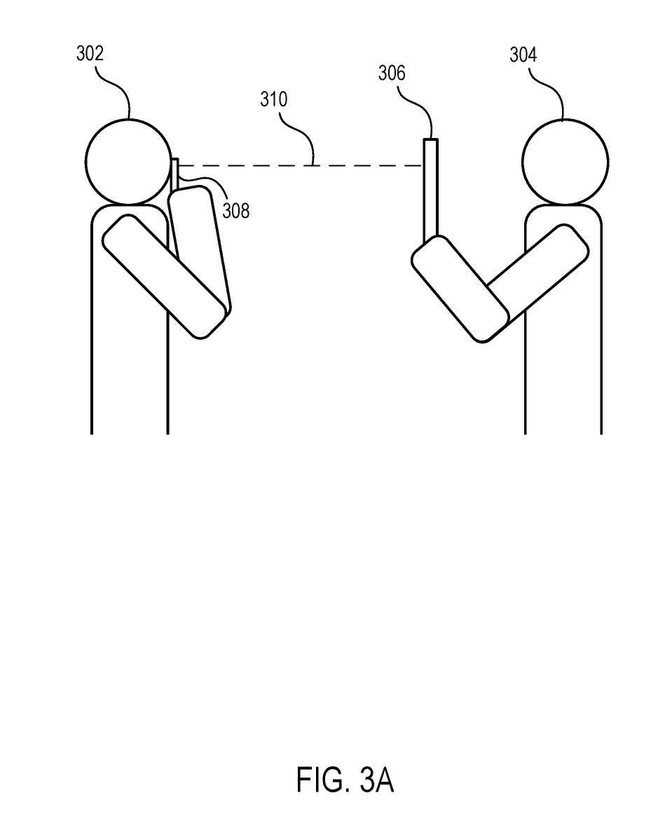

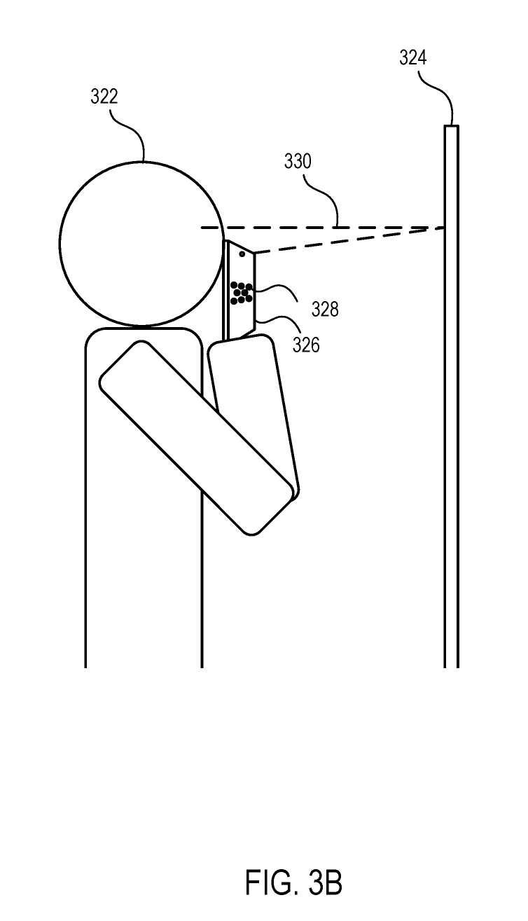

FIG. 3A illustrates an embodiment in which an examiner 304 (e.g., clinician, technician, etc.) positions a user device 306 for a patient 302 while the patient 302 holds a patterned object 308 adjacent to his/her face. Dotted line 310 indicates a line of sight between the patient 302 and a camera of the user device 306.

FIG. 3B illustrates an embodiment in which a patient 322 performs the measurement using a user device 326 as the patterned object. For example, the patient 322 may hold the user device 326 (e.g., smartphone) against his/her face while directing the display screen and camera toward a mirror 324. In certain embodiments, the user device 326 may utilize a built-in gyroscopic sensor to determine if the user device is level and parallel to the vertically-oriented mirror 324. While images are captured, the smartphone may display the pattern 328 on its display screen. The dotted line 330 indicates a line of sight between the patient 322 and the reflected image of the camera. For the purposes of computing a distance from the patient's eyes to the camera, this distance will be taken as twice the distance from the patient's face to the mirror 324, which assumes that the camera is as close to the patient's eyes as possible without obstructing the patient's eyes from view.

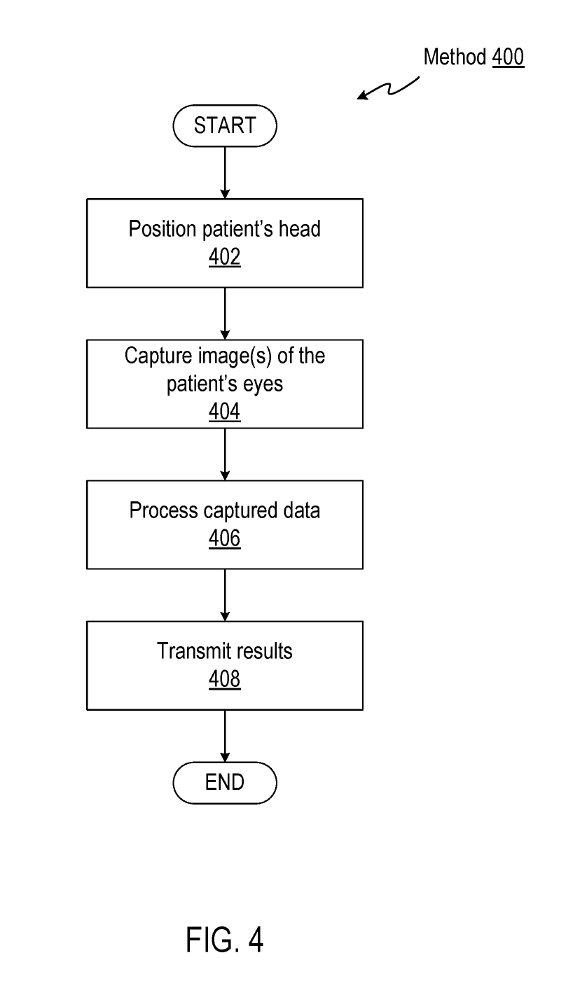

FIG. 4 is a flow diagram illustrating a method 400 for performing a pupillary distance measurement in accordance with various embodiments of the present disclosure. The method 400 starts at block 402, where the patient's head is positioned prior to capturing facial images. In certain embodiments, a patterned object (e.g., a card having a pre-defined pattern thereon) is held, for example, a few inches (e.g., 2 to 5 inches) below the eyes (e.g., in contact with the patient's upper lip). The patient's head posture can introduce tilt that distort the aspect ratio and affect measurement accuracy. In certain embodiments, this may be addressed by having a user device mounted at roughly the same level as the patient's eyes, as illustrated in FIG. 1. Live images may be captured and immediately displayed with graphical overlays on a display screen of the user device to help guide the patient in the head positioning process. In certain embodiments, during real-time image collection, valid images may be separated from invalid images with only valid images being retained for further processing. In certain embodiments that utilize a patterned object, the algorithm may assume that the patient is holding the patterned object as instructed. In such embodiments, the images may be analyzed to determine if the patterned object is present, and if no patterned object is present, the user device may indicate this to the user and may further instruct the user to position the patterned object near his/her face. In certain embodiments, estimation of the distance between patterned object and camera is available to guide the patient's position. For example, on-screen indicators (or audio indicators) may be generated by the user device if the patient is standing too far from or too close to the user device.

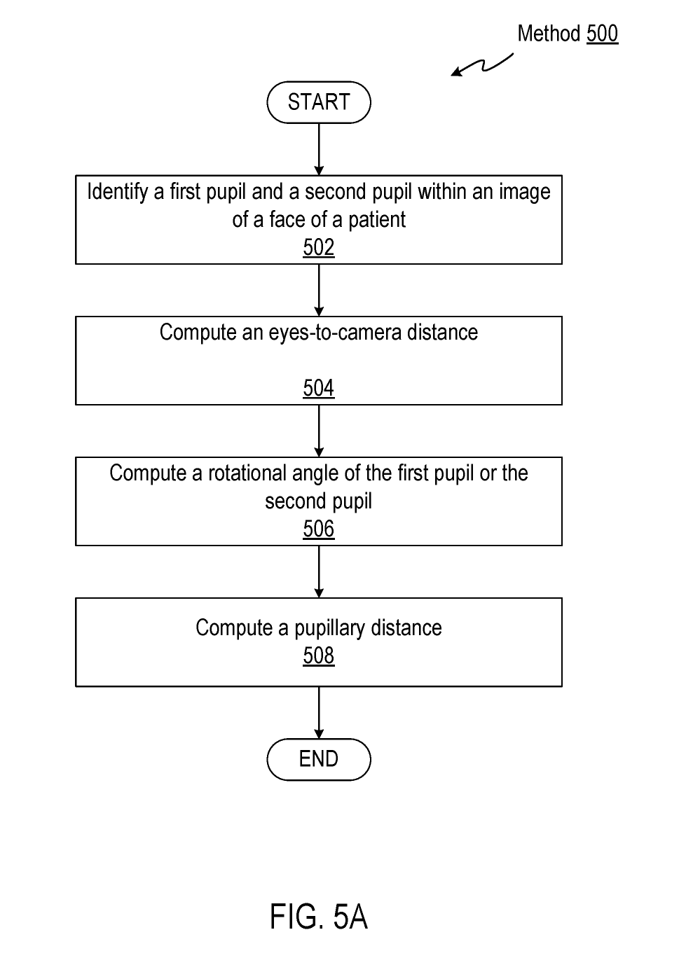

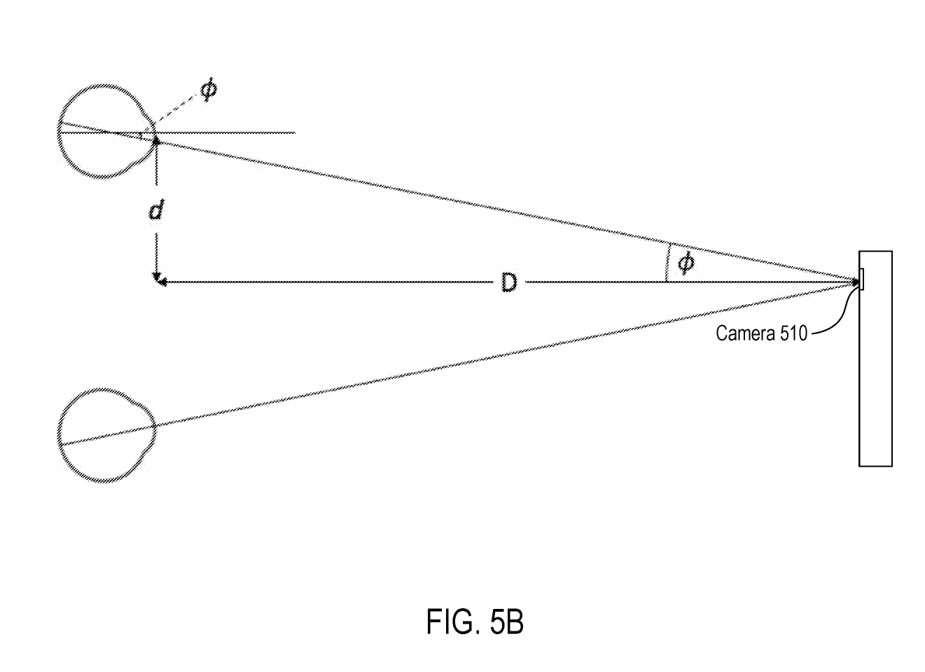

At block 404, one or more images of the patient's eyes are captured. In certain embodiments, image acquisition may last at least 1 second, resulting in multiple images captured depending on the frame rate of the camera (e.g., 30 if the camera is a standard 30-FPS camera). At block 406, the one or more captured images are then processed, for example, as described below with respect any of to FIGS. 5-8. In certain embodiments, patient interaction with the device ends once data collection completes. Once processing is complete, at block 408, results with image verification may be available to publish or transmit to medical personnel.

FIG. 5A is a flow diagram illustrating a method 500 for computing a pupillary distance in accordance with various embodiments of the present disclosure. In certain embodiments, the method 500 is performed by a processing device, such as that of a user device (e.g., the user device 104) or a processing server (e.g., the processing server 110). In certain embodiments, the method 500 commences in response to a determination that a valid facial image was captured.

At block 502, the processing device identifies a first pupil and a second pupil within an image of a face of a patient (e.g., via a segmentation algorithm). In certain embodiments, initially, an area-of-interest (AOI) containing the patient's face is isolated. In certain embodiments, Haar feature-based face detection is utilized to obtain the AOI containing the face, which allows for a smaller number of pixels used in subsequent calculations. Other suitable algorithms may also be used for feature detection in the captured images, such as local binary pattern (LBP) detection algorithms.

In certain embodiments, two sub-AOIs are isolated: one for each eye. Similar to the face detection to identify the initial AOI, a Haar feature-based classifier may be utilized to identify and extract the sub-AOIs containing the patient's eyes. In certain embodiments, the classifier is applied to just the facial AOI rather than to the entire captured image.

In certain embodiments, iris segmentation with the center of the limbus is used to estimate pupil position within each of the two sub-AOIs. The limbic edge may be defined by the outer edge of the iris which interfaces with the sclera. To address the difference in contrast between the iris (which may be relatively dark in the image) and the sclera, the segmentation algorithm, in one embodiment, may utilize an empirically tuned Canny-edge detector with a horizontal Sobel filter followed by contour fitting using Hough circle transforms. The calculations may be performed over pixels within each sub-AOI rather than over the entire captured image. A center-to-center distance measured between the pupils (which is not corrected for eye rotation) is referred to herein as the “near-PD” distance, which may be obtained in units of pixels and converted to physical distance based on calibration discussed below.

At block 504, the processing device computes a distance from the patient's eyes (i.e., a plane defined by the patient's pupils) to a camera used to capture the image (corresponding to the distance between the eyes and the camera at the time that the image was captured). This distance is referred to herein as the “eyes-to-camera distance.”

Various embodiments may be used to compute the eyes-to-camera distance. In certain embodiments, a patterned object (e.g., the patterned object 106) held near the patient's face during image capture may be detectable within the image. In certain embodiments that utilize patterns containing circles, a circle pattern may be detected and its size may be estimated from the number of pixels it spans in the captured image. If the physical size of the pattern is known by the processing device, the eyes-to-camera distance can be derived from the image of the captured pattern, which may also be used to estimate a pixel-to-mm conversion factor for subsequent calculations.

In certain embodiments, the eyes-to-camera distance is computed based on an estimated corneal diameter. Corneal diameter, defined as the diameter of the limbic boundary, is very consistent across the population. The physical diameter of the cornea barely deviates from 11.5 mm, though there is a very weak age dependency which can be accounted for if the patient's age is known (see Hoffmann, P., Hütz, W., “Analysis of biometry and prevalence data for corneal astigmatism in 23239 eyes,” Journal of Cataract & Refractive Surgery, 36(9), 1479-1485, 2010). This anatomical consistency may be exploited by using it as the scaling factor as an alternative to the use of a calibration pattern described in previous embodiments. FIG. 6 illustrates an inner iris boundary 602, an outer iris boundary 604, and a corneal diameter 606. In such embodiments, the segmentation algorithm identifies the outer iris boundary 604 and extracts its diameter, which is taken to be the corneal diameter 606. The corneal diameter 606, in pixels, may then be used as a calibration factor to estimate the near-PD distance and the eyes-to-camera distance.

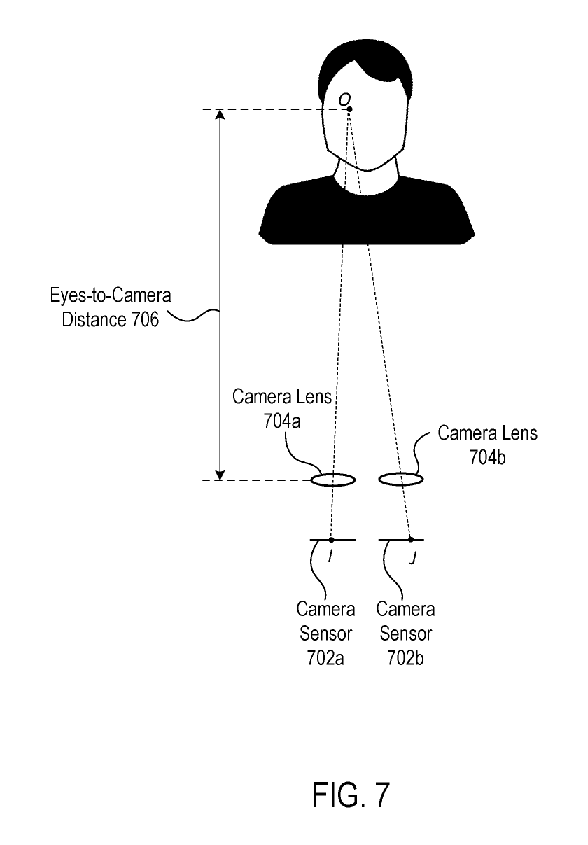

In certain embodiments, the eyes-to-camera distance is computed based on stereo vision computations, which can be performed without the use of a scaling object (e.g., a patterned object, corneal diameter, etc.). Such embodiments may be performed, for example, with a user device that is equipped with dual cameras (e.g. Galaxy Note 8, iPhone 7 Plus, iPhone 8 Plus, etc.). The spacing between the two cameras produces different perspectives of the same object. FIG. 7 illustrates an arbitrary point O (which may correspond to a patient's eye) that is imaged to points I and J on camera sensors 702a and 702b, respectively. Assuming identical cameras with known separation (d), FOV, and pixel support N, the relative pixel difference between points I and J is all can then be used to estimate the eyes-to-camera distance 702 (denoted below as D) according to:

$$D = \frac{d \cdot N}{2 \cdot tan ( \frac{F O V}{2} ) \cdot \overline{IJ}}$$

where I̅J̅ is the relative pixel difference between corresponding image locations of the same object (see Mrovlje, J., Vran, D., “Distance measuring based on stereoscopic pictures,” 9th International PhD Workshop on Systems and Control: Young Generation Viewpoint, 1-6, Oct. 2, 2008). Once distance D is known, the near-PD can be deduced with trigonometric operations applied to one of the two images or the image pair and using an average value.

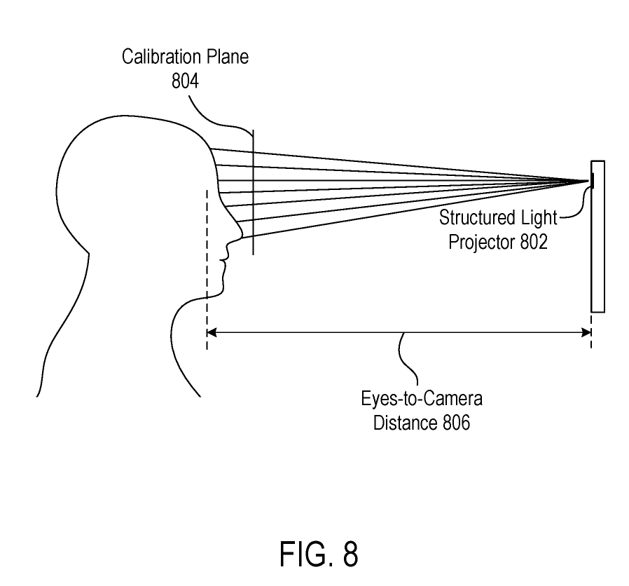

In certain embodiments, the eyes-to-camera distance is computed based on depth information obtained by a user device equipped with structured light imaging systems (e.g., the iPhone X). Capturing an image of an object with a calibrated light pattern projected onto the object allows for three-dimensional property measurements. FIG. 8 illustrates the use of a structured light projector 802. In certain embodiments, the projected light is infrared to avoid harming or irritating the patient's eyes. The camera's FOV fully contains the solid angle of the projected pattern, and the spatial resolution of the light pattern will be sufficient enough to properly sample the pupil area at designated working distances.

The projected pattern may be calibrated on a known surface (e.g., a flat surface) at two or more distances away from the device. Calibration distances may be chosen to lie within the range of expected working distances to minimize error due to physical factors such as diffraction, camera hardware tolerance, and calibration surface tolerance. Proper calibration produces an accurate scaling factor for converting pixels to physical distance as a function of working distance (e.g., eyes-to-camera distance 808). Relevant structured light geometry (e.g., for iPhone X spot projector, it is the beam launch angle for each of the 30,000 spots) is also obtained during calibration since both object distance and camera focal length are known. The pattern produced by the structured light over the pixels defining the iris areas are analyzed and referenced against calibration data to estimate working distance. The specific algorithm for this image processing step depends on structured light design. For the spot projector example, an algorithm would identify the relevant spot locations in the image, match them up to the corresponding calibration reference spot locations, compute the difference to estimate relative shift from a calibration plane 804, and add the result to that of the predefined distance between calibration and camera planes.

Referring once again to FIG. 5A, at block 506, the processing device computes a rotational angle of the first pupil or the second pupil (e.g., based on the eyes-to-camera distance and the rotational angle). Assuming that the patient's eyes are healthy, the patient's eyes will rotate nasally for near viewing, and may rotate significantly when the camera is placed within a few feet of the patient's face. In certain embodiments, to accurately estimate pupillary distance from near-PD distance, the rotation may be calibrated out. The aperture and detector size of the camera is used to determine its FOV, which allows for estimates of the distance from the patient's eyes or other objects to the camera (with the assumption that the patterned object is “in-plane” with the patient's face). In some embodiments, distance from the pupil plane to the retina is estimated to be 20.37 mm. Assuming that the eye rotates from the center of that distance, similar triangles analysis may be used to determine the rotation angle, and the corresponding linear pupil shift may then be added to the near-PD estimate to arrive at the final pupillary distance measurement. FIG. 5B illustrates calculation of eye rotational angle (ϕ) using similar triangle analysis, where ϕ can be estimated once the near-PD distance is known. Both near-PD (2d) and the distance between the camera 510 and pupil plain (D) may be determined via image processing. The angle θ used estimate the pupillary distance from the near-PD distance requires angle is computed as the arctangent of d/D. The embodiments described herein allow for computation of the pupillary distance between anatomical extremes (e.g., between 52 and 76 mm).

It is to be understood by one of ordinary skill in the art that one or more of the features of the described methods may be performed in a different order than discussed or shown, that one or more of the features of a particular method may be omitted or combined with another, or that one or more features of different methods may be combined to yield variations of the described methods. In certain embodiments, the flow of the methods are implemented as a rejection cascade, with each feature being performed sequentially as discussed above with the final pupillary distance measurement being computed after successful completion of all prior features.

### General Computer System Embodiments

FIG. 9 illustrates a diagrammatic representation of a machine in the exemplary form of a computer system 900 within which a set of instructions (e.g., for causing the machine to perform or facilitate performance of any one or more of the methodologies discussed herein) may be executed. In alternative embodiments, the machine may be connected (e.g., networked) to other machines in a LAN, an intranet, an extranet, or the Internet. The machine may operate in the capacity of a server or a client machine in client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Some or all of the components of the computer system 900 may be utilized by or illustrative of any of the processing devices described herein (e.g., a processing device of the user device 104), user devices (e.g., user device 104), or any other devices that may send/receive information to/from any of the devices described herein.

The exemplary computer system 900 includes a processing device (processor) 902, a main memory 904 (e.g., read-only memory (ROM), flash memory, dynamic random access memory (DRAM) such as synchronous DRAM (SDRAM) or Rambus DRAM (RDRAM), etc.), a static memory 906 (e.g., flash memory, static random access memory (SRAM), etc.), and a data storage device 920, which communicate with each other via a bus 910.

Processor 902 represents one or more general-purpose processing devices such as a microprocessor, central processing unit, or the like. More particularly, the processor 902 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 902 may also be one or more special-purpose processing devices such as an application specific integrated circuit (ASIC), a field programmable gate array (FPGA), a digital signal processor (DSP), network processor, or the like. The processor 902 is configured to execute instructions 926 for performing the operations and steps discussed herein.

The computer system 900 may further include a network interface device 908. The computer system 900 also may include a video display unit 912 (e.g., a liquid crystal display (LCD), a cathode ray tube (CRT), or a touch screen), an alphanumeric input device 914 (e.g., a keyboard), a cursor control device 916 (e.g., a mouse), and a signal generation device 922 (e.g., a speaker).

Power device 918 may monitor a power level of a battery used to power the computer system 900 or one or more of its components. The power device 918 may provide one or more interfaces to provide an indication of a power level, a time window remaining prior to shutdown of computer system 900 or one or more of its components, a power consumption rate, an indicator of whether computer system is utilizing an external power source or battery power, and other power related information. In certain embodiments, indications related to the power device 918 may be accessible remotely (e.g., accessible to a remote back-up management module via a network connection). In certain embodiments, a battery utilized by the power device 918 may be an uninterruptable power supply (UPS) local to or remote from computer system 900. In such embodiments, the power device 918 may provide information about a power level of the UPS.

The data storage device 920 may include a computer-readable storage medium 924 on which is stored one or more sets of instructions 926 (e.g., software) embodying any one or more of the methodologies or functions described herein. The instructions 926 may also reside, completely or at least partially, within the main memory 904 and/or within the processor 902 during execution thereof by the computer system 900, the main memory 904 and the processor 902 also constituting computer-readable storage media. The instructions 926 may further be transmitted or received over a network 930 via the network interface device 908.

In one embodiment, the instructions 926 include instructions for performing various electronic operations, such as processing image data and/or controlling the operation of components within an optical device. While the computer-readable storage medium 924 is shown in an exemplary embodiment to be a single medium, the terms “computer-readable storage medium” or “machine-readable storage medium” should be taken to include a single medium or multiple media (e.g., a centralized or distributed database, and/or associated caches and servers) that store the one or more sets of instructions. The terms “computer-readable storage medium” or “machine-readable storage medium” shall also be taken to include any transitory or non-transitory medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

Some portions of the detailed description may have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is herein, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities.

Unless specifically stated otherwise as apparent from the preceding discussion, it is appreciated that throughout the description, discussions utilizing terms such as “receiving,” “storing,” “transmitting,” “computing,” “processing,” “indicating,” “capturing,” “identifying,” “detecting,” “segmenting,” “estimating,” “analyzing,” “generating,” “displaying,” “rendering for display,” “activating,” “deactivating,” “controlling,” or the like, refer to the actions and processes of a computer system, or similar electronic computing device. The actions may be used by the computer system, or similar electronic computing device, to manipulate and transform data represented as physical (e.g., electronic) quantities within the computer system's registers and memories into other data similarly represented as physical quantities within the computer system memories or registers or other such information storage, transmission, or display devices. The actions may also be used by the computer system, or similar electronic computing device, to control the operation of other electronic devices.

Certain embodiments of the disclosure relate to an apparatus, device, or system for performing the operations herein. This apparatus, device, or system may be specially constructed for the required purposes, or it may include a general purpose computer selectively activated or reconfigured by a computer program stored in the computer. Such a computer program may be stored in a computer- or machine-readable storage medium, such as, but not limited to, any type of disk including floppy disks, optical disks, compact disk read-only memories (CD-ROMs), and magnetic-optical disks, read-only memories (ROMs), random access memories (RAMs), erasable programmable read-only memories (EPROMs), electrically erasable programmable read-only memories (EEPROMs), magnetic or optical cards, or any type of media suitable for storing electronic instructions.

For simplicity of explanation, the methods of the present disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. Additionally, it should be appreciated that algorithms disclosed in this specification are capable of being stored on an article of manufacture to facilitate transporting and transferring such algorithms to computing devices for execution. The term “article of manufacture”, as used herein, is intended to encompass a computer program accessible from any computer-readable device or storage media.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the example embodiments that follow should generally be construed to mean “one or more” unless specified otherwise or clear from context to be directed to a singular form. Reference throughout this specification to “an embodiment” or “one embodiment” means that a particular feature, structure, or characteristic described in connection with the embodiment is included in at least one embodiment. Thus, the appearances of the phrase “an embodiment” or “one embodiment” in various places throughout this specification are not necessarily all referring to the same embodiment.

It is to be understood that the above description is intended to be illustrative, and is not intended to be limited by the specific embodiments described herein or by way of illustration in the accompanying drawings. Indeed, other various embodiments of and modifications to the present disclosure, in addition to those described herein, will be apparent to those of ordinary skill in the art from the preceding description and accompanying drawings. Thus, such other embodiments and modifications pertaining to optical analysis of a patient's eye are intended to fall within the scope of the present disclosure. Further, although the present disclosure has been described herein in the context of particular embodiments in particular environments for particular purposes, those of ordinary skill in the art will recognize that its usefulness is not limited thereto and that the present disclosure may be beneficially implemented in any number of environments for any number of purposes. Accordingly, the example embodiments set forth below should be construed in view of the full breadth and spirit of the present disclosure as described herein, along with the full scope of equivalents to which such embodiments are entitled.

What is claimed is:

1. A method for measuring a pupillary distance of a patient, the method comprising:
identifying, by a processing device, a first pupil and a second pupil within an image of a face of the patient;
computing, by the processing device, an eyes-to-camera distance corresponding to a distance from a plane defined by the first pupil and the second pupil to a camera used to capture the image at a time of the image capture;
computing, by the processing device, a rotational angle of the first pupil or the second pupil; and
computing, by the processing device, the pupillary distance based on the eyes-to-camera distance and the rotational angle.

2. The method of claim 1, wherein identifying the first pupil and the second pupil within the image comprises:
detecting a first area-of-interest within the image, the first area-of-interest corresponding to the face of the patient;
detecting a second area-of-interest within the first area-of-interest, the second area-of-interest comprising an upper portion of the face comprising a first eye and a second eye;
detecting third and fourth areas-of-interest within the second area-of-interest, the third and fourth areas-of-interest corresponding to the first eye and the second eye, respectively; and
identifying the first pupil and the second pupil within the third and fourth areas-of-interest, respectively.

3. The method of claim 1, further comprising:
identifying and isolating pixels within the image that correspond to a pattern of a patterned object, wherein the eyes-to-camera distance is computed based on the isolated pixels.

4. The method of claim 1, further comprising:
identifying and isolating pixels within the image that correspond to a cornea of the patient, wherein the eyes-to-camera distance is computed by calibrating the image based on an estimated diameter of the cornea.

5. The method of claim 1, wherein the eyes-to-camera distance is computed based on a stereo vision computation using an additional image of the face of the patient captured by an additional camera substantially simultaneously with the capture of the first image.

6. The method of claim 1, wherein the eyes-to-camera distance is computed based on depth information associated with the capture of the first image, wherein the depth information is derived from a light pattern projected onto the patient's face during the capture of the first image.

7. The method of claim 1, wherein computing the rotational angle of the first pupil or the second pupil comprises:
computing a near-PD distance from the image; and
computing the rotational angle as the arctangent of a ratio of the near-PD distance to the eyes-to-camera distance.

8. A system for measuring a pupillary distance of a patient, the system comprising:
a memory;
a processing device communicatively coupled to the memory, wherein the processing device is configured to:
identify a first pupil and a second pupil within an image of a face of the patient;
compute an eyes-to-camera distance corresponding to a distance from a plane defined by the first pupil and the second pupil to a camera used to capture the image at a time of the image capture;
compute a rotational angle of the first pupil or the second pupil;
compute the pupillary distance based on the eyes-to-camera distance and the rotational angle; and
store the computed pupillary distance in the memory.

9. The system of claim 9, wherein to identify the first pupil and the second pupil within the image, the processing device is further configured to:
detect a first area-of-interest within the image, the first area-of-interest corresponding to the face of the patient;
detect a second area-of-interest within the first area-of-interest, the second area-of-interest comprising an upper portion of the face comprising a first eye and a second eye;
detect third and fourth areas-of-interest within the second area-of-interest, the third and fourth areas-of-interest corresponding to the first eye and the second eye, respectively; and
identify the first pupil and the second pupil within the third and fourth areas-of-interest, respectively.

10. The system of claim 9, wherein the processing device is further configured to:
identify and isolate pixels within the image that correspond to a pattern of a patterned object, wherein the eyes-to-camera distance is computed based on the isolated pixels.

11. The system of claim 9, wherein the processing device is further configured to:
identify and isolate pixels within the image that correspond to a cornea of the patient, wherein the eyes-to-camera distance is computed by calibrating the image based on an estimated diameter of the cornea.

12. The system of claim 9, wherein the eyes-to-camera distance is to be computed based on a stereo vision computation using an additional image of the face of the patient captured by an additional camera substantially simultaneously with the capture of the first image.

13. The system of claim 9, wherein the eyes-to-camera distance is to be computed based on depth information associated with the capture of the first image, wherein the depth information is derived from a light pattern projected onto the patient's face during the capture of the first image.

14. The system of claim 9, wherein to compute the rotational angle of the first pupil or the second pupil, the processing device is further configured to:
compute a near-PD distance from the image; and
compute the rotational angle as the arctangent of a ratio of the near-PD distance to the eyes-to-camera distance.

15. A non-transitory computer-readable storage medium having instructions stored thereon that, when executed by a processing device, cause the processing device to perform operations comprising:
identifying a first pupil and a second pupil within an image of a face of the patient;
computing an eyes-to-camera distance corresponding to a distance from a plane defined by the first pupil and the second pupil to a camera used to capture the image at a time of the image capture;
computing a rotational angle of the first pupil or the second pupil; and
computing the pupillary distance based on the eyes-to-camera distance and the rotational angle.

16. The non-transitory computer-readable storage medium of claim 15, wherein the operations further comprise:
detecting a first area-of-interest within the image, the first area-of-interest corresponding to the face of the patient;
detecting a second area-of-interest within the first area-of-interest, the second area-of-interest comprising an upper portion of the face comprising a first eye and a second eye;
detecting third and fourth areas-of-interest within the second area-of-interest, the third and fourth areas-of-interest corresponding to the first eye and the second eye, respectively; and
identifying the first pupil and the second pupil within the third and fourth areas-of-interest, respectively;
computing a near-PD distance based on the third and fourth areas-of-interest; and
computing the rotational angle as the arctangent of a ratio of the near-PD distance to the eyes-to-camera distance.

17. The non-transitory computer-readable storage medium of claim 15, wherein the operations further comprise:
identifying and isolating pixels within the image that correspond to a pattern of a patterned object, wherein the eyes-to-camera distance is computed based on the isolated pixels.

18. The non-transitory computer-readable storage medium of claim 15, wherein the operations further comprise:
identifying and isolating pixels within the image that correspond to a cornea of the patient, wherein the eyes-to-camera distance is computed by calibrating the image based on an estimated diameter of the cornea.

19. The non-transitory computer-readable storage medium of claim 15, wherein the eyes-to-camera distance is to be computed based on a stereo vision computation using an additional image of the face of the patient captured by an additional camera substantially simultaneously with the capture of the first image.

20. The non-transitory computer-readable storage medium of claim 15, wherein the eyes-to-camera distance is to be computed based on depth information associated with the capture of the first image, wherein the depth information is derived from a light pattern projected onto the patient's face during the capture of the first image.
